(19) United States
(12) Patent Application Publication
(10) Pub. No.: US 2012/0259171 A1
(43) Pub. Date: Oct. 11, 2012

(54) Entertainment System and Device

(76) Inventor: Andrey A. SHMAKOV, St. Petersburg (RU)

(21) Appl. No.: 13/528,957

(22) Filed: Jun. 21, 2012

Related U.S. Application Data
(63) Continuation of application No. PCT/IB2010/003514, filed on Dec. 23, 2010.
(60) Provisional application No. 61/289,970, filed on Dec. 23, 2009.

(51) Int. Cl. A61F 5/00; H04B 1/38

(52) U.S. Cl. 600/38

(57) ABSTRACT

A console comprising attachments including sex toys and a network interface. A system for communication between consoles and a central server to allow users to participate in a variety of types of remote sex.

### RELATED APPLICATIONS

This application is a continuation of International Application No. PCT/IB2010/003514, filed Dec. 23, 2010, which claims the benefit of U.S. Provisional Application No. 61/289,670, filed Dec. 23, 2009, which is incorporated herein by reference in its entirety.

### BACKGROUND OF THE INVENTION

A variety of devices simulating sexual intercourse and other kinds of sexual stimulation is available on the market: vibrating dildos and artificial vaginas, sex dolls with mechanical capabilities, etc.

The present invention relates to using computer networks (such as Internet) for providing and delivering sexual and erotic experiences similar to the experiences provided by the aforementioned devices.

### SUMMARY OF THE INVENTION

The system comprises at least one console comprising a network interface capable of sending and receiving commands and information over a computer network; a memory unit storing user's preferences; attachments comprising at least one sex toy; a transceiver for communicating with the attachments; a controller connected with the network interface, the memory unit, and the transceiver.

The system may further comprise a central server communicating with the consoles to establish user sessions. The attachments may comprise a wireless dildo; a wired dildo; a wireless artificial vagina; a wired artificial vagina; a projector; a sensor; a helmet; a patch; and/or a base for installing other attachments.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 shows the consoles with various attachments connected to the network via personal computers and a mobile phone and communicating with the server.

FIG. 2 shows a console.

### DETAILED DESCRIPTION OF THE PREFERRED EMBODIMENTS

The present system permits erotic and sexual interactions between two or more users over computer networks using the consoles described below.

These uses may form a club for remote sexual interactions (involving two or more participants) in real time or using stored data.

The participants may have one or more alter egos (avatars) in this club.

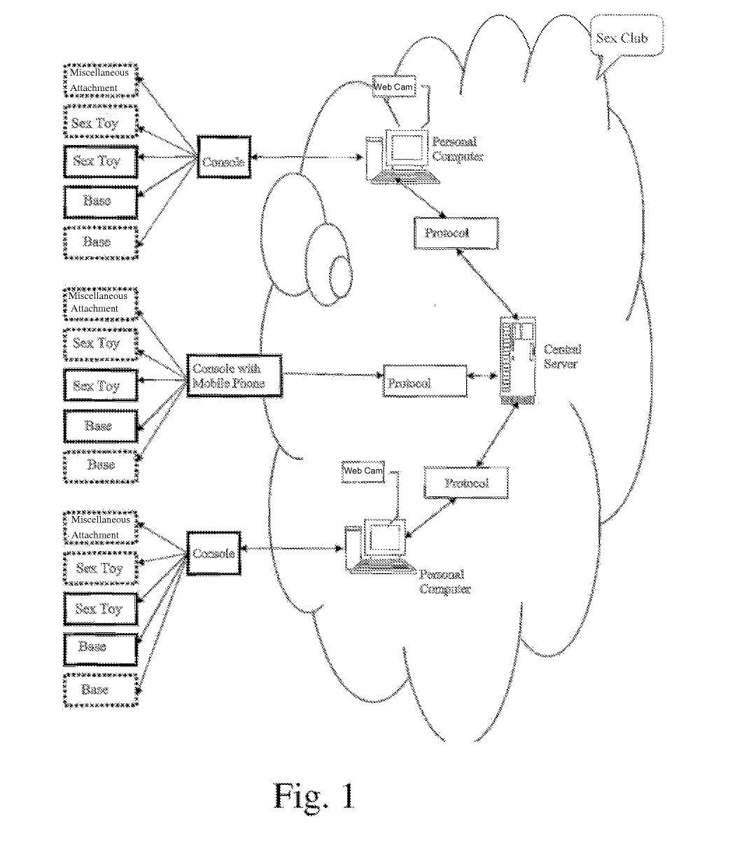

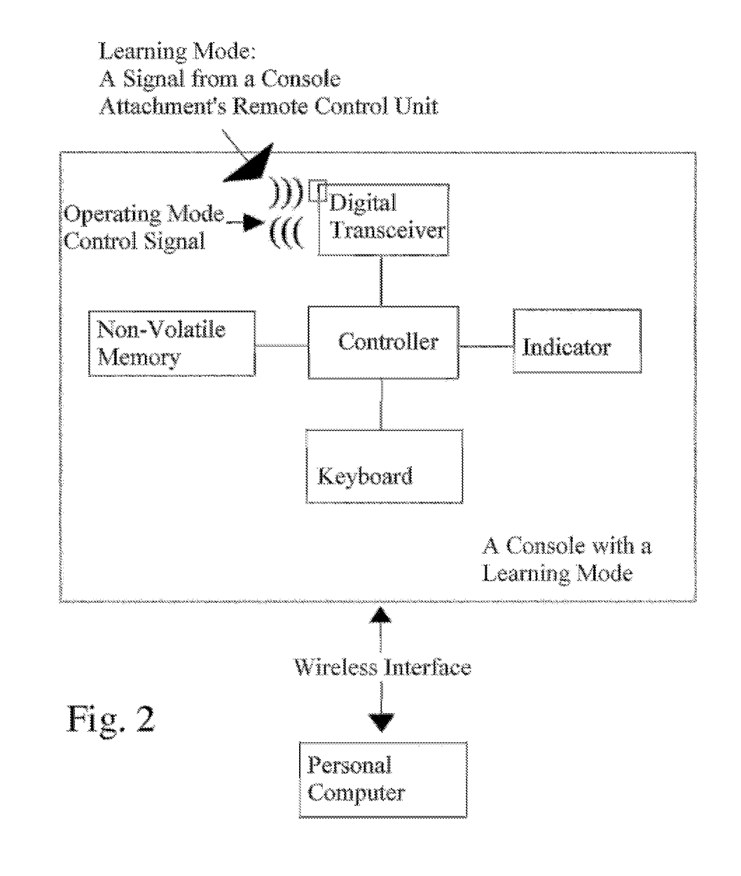

The system as shown in FIG. 1 uses a console with console attachments controlled by a controller in the console. The console, shown in FIG. 2, in addition to the controller, comprises a transceiver for communicating with attachments, a memory unit, an indicator to show the console's status, and a keyboard for entering data. The network interface of the console allows connecting to a network computer, mobile phone connected to a computer network, or directly to a computer network. The controller may be capable of storing individual settings for each attachment.

The console attachments may include any variety of sex toys capable of being controlled by the controller, such as active sex toys with independent power supply and wireless remote control for stimulating a variety of body parts of males and females in a variety of ways.

Another type of console attachment is a base in a shape of a lounger, a bed, or a human body to which sex toys may be installed. This base may be adjusted for size, physical capabilities and sexual preferences of its user. A user may use several bases simultaneously, for example, for simultaneous simulation of oral, vaginal, and anal sex. The base may be made adjustable so that the use may adjust the angle, strength, depth, and frequency of vibration or penetration. The base may include blocks of elements, such as an anal-vaginal block with separate actuators for anal and vaginal stimulation or simulation.

The sex toys may be installed on the base, attached to the body, or held by the user.

Each sex toy may have several settings controlling its actions (gradually or not) and each setting may be actuated by the controller and/or adjusted by the user. The information about the presets may be stored in the console's memory.

A sex toy may be controlled solely by its user alone without interacting with other users of the system. A sex toy may also be controlled by a remote user through a computer network.

A sex toy's dynamics may also be coordinated with the dynamics of another sex toy of another user. A user may send a synchronizing signal to his or her sex toy and a corresponding sex toy of his or her partner gets activated. Or a user (through his or her console) may send a signal to the partner's console and the corresponding user's sex toy gets activated.

Another kind of console attachments, referred to as miscellaneous attachments, includes devices for providing tactile, physical, psychological, visual, sound, light and other effects for the user. They may include projectors, generators, sensors, helmets, patches, and wearable devices. These devices may be controlled in the system in the same way as the sex toy attachments.

The computer and gaming consoles may incorporate web cameras, microphones, and sound devices.

The console sends control signals to the attachments and communicates with a computer, gaming device, another network device, or directly to the network. The commands to the miscellaneous attachments may arrive to the console from the user, or his computer communicating with other consoles of the club members.

For communicating with attachments, computers, and cell phones, the console may use wired communications as well as wireless communications (Bluetooth, Wi-Fi, WiMAX, radio communication, etc.) The console may include graphic user interface, which may provide visual status for console attachments used in the session.

A console has a learning mode, wherein the drivers and settings for the attachments are determined and preferences are set. The drivers may be provided by the central server. In the learning mode, the correspondence between keys on the console and the attachment functions is established.

A console also has a work mode wherein the central server sends control signals to the console. A console also has an autonomous mode wherein all control over the attachments is provided through the console's interface, which includes for example, keyboard and a screen.

The consoles are used as a part of the network system which includes a central server maintaining a list of users, providing billing, establishing sessions between consoles, sending signals to attachments through the consoles using a network protocol for communications between the central server and the consoles of the club members. The central server may also provide language assistance to club members using different languages.

The system functions as a sex club for members using the consoles. The users may advertise to other users their availability for a session using a club's bulletin board, to ask for permission to participate in existing sessions, to watch ongoing sessions as a spectator, to offer oneself as a “sex slave”, or hire a “sex slave”. This participation may be on behalf of the user himself or on behalf of the user's avatar.

The sessions may be one-on-one active sessions, group, one-on-one mater/slave sessions, or passive sessions where one of the participants is a spectator. The availability and participation of users in the sessions may be determined by the users according to their conditions and preferences and also depending on the capabilities of their consoles.

Before initiating a session the users would send (via their consoles) the parameters of the session in which they would want to participate. The central server would send appropriate settings to the consoles (to set the attachments mode) and perform the necessary billing and registration operations.

Mutual active one-on-one sessions would permit the participants (of the same or of the opposite sexes) to experience remote sex using the console attachments. The roles would be determined by the participants in advance, including determining who of the participants would be an active participant and who would be a passive participant. The active participant would set the initial control signals for the attachments of the participants. Each console may function in the active mode (sending control signals) and in the passive mode (receiving control signals) simultaneously.

Therefore, three situations are possible: (a) an attachment is not synchronized with any other participant, and the user controls the attachment himself, (b) one active user controls the attachment on the console belonging to another passive user, or (c) the console attachments of both users are synchronized and the active user sets their modes. The variant (c) permits two realizations: the active user sends a signal to his own console attachment and the passive partner's console attachment receives a synchronizing signal or the active user sends a control signal to the console of the partner and receives a response signal to his console attachment.

The commands are sent as following:

In the (b) mode the active user sends a command to control the dynamics of the devices belonging to the passive user. This signal from the console belonging to the active user is sent to his computer and then, through the computer network and the central server to the passive user's computer, then to the passive user's console, and finally to the console attachment.

In the (c) mode the active user uses his console to send control signal to two synchronized devices (his own and his partner's) simultaneously. The partner's console and both computers serve as re-translators and transmitters of the initial control signal. The video and audio link may be established between the partners as well. The synchronization would permit for example coordination of dildo dynamics for one user and the dynamics of artificial vagina for another user. In “open” sessions other users may participate as spectators or observers.

The group sessions are similar to one-on-one sessions but allow a plurality of participants who may join or disconnect from the session at the time of their choosing. The users would inform other participants in the group session regarding availability of their console attachment for remote control and synchronization. Some of the participants may be paid actors.

The users may also create avatars on the central server so that other users may enjoy virtual sex with the avatars. The group sessions may be joined by avatars as well as by real persons.

The session of a real user or of an avatar may be recorded for delayed use.

While this invention has been particularly shown and described with references to preferred embodiments thereof, it will be understood by those skilled in the art that various changes in form and details may be made therein without departing from the scope of the invention encompassed by the appended claims.

What is claimed is:

1. A system comprising at least one console comprising:
a network interface capable of sending and receiving commands and information over a computer network;
a memory unit storing user's preferences;
attachments comprising at least one sex toy;
a transceiver for communicating with the attachments; and
a controller connected with the network interface, the memory unit, and the transceiver.

2. The system of claim 1 further comprising
a central server communicating with the consoles to establish user sessions.

3. The system of claim 1 wherein the attachments comprise at least one of:
a wireless dildo;
a wired dildo;
a wireless artificial vagina;
a wired artificial vagina;
a projector;
a sensor;
a helmet;
a patch;
a base for installing other attachments.
